(19) United States
(12) Patent Application Publication
(10) Pub. No.: US 2015/0160240 A1
(43) Pub. Date: Jun. 11, 2015

(54) Host Biomarkers for Dengue Fever (DF) and Methods Thereof

(71) Applicants: NATIONAL UNIVERSITY OF SINGAPORE, Singapore (SG); DUKE UNIVERSITY, Durham, NC (US)

(72) Inventors: Soman Ninan Abraham, Durham, NC (US); Ashley Lauren St. John, Durham, NC (US); Mary Mah Lee Ng, Singapore (SG); Bhuvanakantham Raghavan, Singapore (SG)

(21) Appl. No.: 14/387,974

(22) PCT Filed: Mar. 15, 2013
(86) PCT No.: PCT/US2013/032265

§ 371 Date: Sep. 25, 2014

Related U.S. Application Data
(60) Provisional application No. 61/616,062, filed on Mar. 27, 2012.
(60) Provisional application No. 61/677,041, filed on Jul. 30, 2012.

(51) Int. Cl. G01N 33/68; G01N 33/573; G01N 33/74

(52) U.S. Cl. CPC G01N 33/6893; G01N 33/74; G01N 33/573; G01N 2800/26; G01N 2333/96433; G01N 2333/4713; G01N 2333/475

(57) ABSTRACT

The present invention provides methods of determining whether a subject has a Dengue virus infection that is likely to develop into a life-threatening Dengue disease and methods of selecting a treatment regimen for a subject in need thereof who has been infected with a Dengue virus. The invention further provides a prognostic kit for distinguishing a subject who is likely to develop a mild Dengue disease from a subject who is likely to develop a life-threatening Dengue disease. The methods and kits of the invention can be used to differentiate DHF from DF and severe and non-severe forms of Dengue with high sensitivity and specificity.

### RELATED APPLICATIONS

This application claims the benefit of U.S. Provisional Application No. 61/616,062, filed on Mar. 27, 2012 and U.S. Provisional Application No. 61/677,041, filed on Jul. 30, 2012. The entire teachings of the above applications are incorporated herein by reference.

### BACKGROUND OF THE INVENTION

Dengue is the most medically important arboviral disease in humans. Its etiological agent, the Dengue virus (DENV), causes a wide range of diseases, ranging from acute febrile Dengue fever (DF) to life-threatening Dengue hemorrhagic fever (DHF) and Dengue shock syndrome (DSS).

Dengue fever is self-limited though debilitating illness characterized by fever, frontal headache, retro-orbital pain, myalgia, arthralgia, nausea, vomiting, weakness and rash. Dengue hemorrhagic fever is marked by increased vascular permeability, thrombocytopenia and hemorrhagic manifestations. Common hemorrhagic manifestations include skin hemorrhages such as petechiae, purpuric lesions and ecchymoses. Epistaxis, bleeding gums, gastro-intestinal hemorrhage and hematuria occur less frequently. Dengue shock syndrome occurs when fluid leakage into the interstitial spaces results in shock, which without appropriate treatment may lead to death [reviewed from (1-3)].

The frequency and magnitude of epidemic Dengue have increased dramatically in the past 40 years as the viruses and the mosquito vectors have both expanded geographically in the tropical regions of the world (4). Over half of the world's population (3.6 billion) is at risk of DENV infection, with an estimated 34 million cases of clinical DF, 2 million cases of DHF and over 20,000 deaths each year (4). Although these viruses account for several million human infections annually, there are no effective therapeutic options currently available. The most effective protective measures at this moment include vector control program and personal protective measures. However, vector control strategies failed to prevent the emergence of Dengue epidemics. Hence, physicians can only rely on early recognition and prompt supportive treatment to lower the risk of developing severe disease complications such as DHF/DSS. However, there is a clear lack of prognostic biomarkers to accurately identify patients who will develop DHF/DSS. Dengue patients are hospitalized on a first-come-first-serve basis and this led to a great shortage of hospital beds and significant burden on the healthcare infrastructure during major Dengue epidemics. Hence, there is an urgent need to identify effective prognostic biomarkers to differentiate severe Dengue (DHF/DSS) from mild febrile illness (DF).

### SUMMARY OF THE INVENTION

The present invention provides, in one embodiment, a method of determining whether a subject in need thereof has a Dengue virus infection that is likely to develop into a life-threatening Dengue disease. The method of this embodiment comprises (a) detecting in a sample from the subject an expression level of at least two proteins selected from the group consisting of vascular endothelial growth factor protein (SEQ ID NO:1), chymase-1 protein (SEQ ID NO:2) and alpha-2 macroglobulin protein (SEQ ID NO:3); (b) comparing the expression level of each protein detected in (a) to a corresponding expression level for each protein in a control; and (c) determining that the patient has a Dengue virus infection that is likely to develop into a life-threatening Dengue disease when the expression level of each protein detected in the sample in (a) is significantly greater than the corresponding expression level for each protein in the control. In a particular embodiment, the method comprises detecting an expression level of vascular endothelial growth factor protein (SEQ ID NO:1), chymase-1 protein (SEQ ID NO:2) and alpha-2 macroglobulin protein (SEQ ID NO:3) in the sample from the subject.

In another embodiment, the invention relates to a method of determining whether a subject in need thereof has a Dengue virus infection that is likely to develop into a life-threatening Dengue disease comprising (a) detecting in a sample from the subject an expression level of at least two proteins selected from the group consisting of vascular endothelial growth factor protein (SEQ ID NO:1), chymase-1 protein (SEQ ID NO:2) and alpha-2 macroglobulin protein (SEQ ID NO:3); (b) comparing the expression level of each protein detected in (a) to a corresponding expression level for each protein in a control; (c) determining that the patient has a Dengue virus infection that is likely to develop into a life-threatening Dengue disease when the expression level of each protein detected in the sample in (a) is significantly greater than the corresponding expression level for each protein in the control; and (d) administering a treatment regimen for a life-threatening Dengue disease to the subject upon determining that the subject has a Dengue virus infection that is likely to develop into a life-threatening Dengue disease. In a particular embodiment, the treatment regimen is administered to the subject before the onset of symptoms of life-threatening Dengue disease.

In a further embodiment, the invention relates to a method of selecting a treatment regimen for a subject in need thereof who has been infected with a Dengue virus, comprising (a) detecting in a sample from the subject an expression level of at least two proteins selected from the group consisting of vascular endothelial growth factor protein (SEQ ID NO:1), chymase-1 protein (SEQ ID NO:2), and alpha-2 macroglobulin protein (SEQ ID NO:3); (b) comparing the expression level of each protein detected in (a) to a corresponding expression level for each protein in a control; and (c) selecting either: (1) a regimen for treating a life-threatening Dengue disease when the expression level of each protein detected in the sample in (a) is significantly greater than the corresponding expression level for each protein in the control; or (2) a regimen for treating a mild Dengue disease when the expression level of each protein detected in the sample in (a) is not significantly greater than the corresponding expression level for each protein in the control. In a particular embodiment, the method further comprises administering the selected regimen to the subject.

In yet another embodiment, the invention relates to a prognostic kit for distinguishing a subject who is likely to develop a mild Dengue disease from a subject who is likely to develop a life-threatening Dengue disease. The kit comprises at least two of: (a) one or more reagents for detecting an expression level of: (1) a vascular endothelial growth factor protein, (2) an antibody to a vascular endothelial growth factor protein, or (3) a combination thereof; (b) one or more reagents for detecting an expression level of: (1) a chymase-1 protein, (2) an antibody to a chymase-1 protein, or (3) a combination thereof or (c) one or more reagents for detecting an expression level of: (1) an alpha-2 macroglobulin protein, (2) an antibody to an alpha-2 macroglobulin protein, or (3) a combination thereof.

The methods and kits of the invention can be used to differentiate DHF from DF and severe and non-severe forms of Dengue with high sensitivity and specificity.

### BRIEF DESCRIPTION OF THE DRAWINGS

FIG. 1A is a graph depicting levels of VEGF protein in serum samples obtained from Dengue patients with different degrees of severity. VEGF is significantly increased in DHF samples compared to that of DF at the early time points.

FIG. 1B is a graph depicting levels of chymase-1 protein in serum samples obtained from Dengue patients with different degrees of severity. Chymase levels are significantly increased in DHF samples compared to that of DF at the early time points.

FIG. 2A is a graph depicting levels of alpha-2 macroglobulin protein in serum samples from Dengue patients with different degrees of severity obtained from a prospective study (first batch of sera). Significantly higher levels of macroglobulin are observed in DHF samples compared to that of DF samples at both early and late time points.

FIG. 2B is a graph depicting levels of chymase-1 protein in serum samples from Dengue patients with different degrees of severity obtained from a prospective study (first batch of sera). Chymase levels are significantly increased in DHF sera samples only at the early time point.

FIG. 2C is a graph depicting levels of VEGF protein in serum samples from Dengue patients with different degrees of severity obtained from a prospective study (first batch of sera). No significant differences in the levels of VEGF between DF and DHF serum samples are obtained.

FIG. 3A is a graph depicting levels of chymase-1 protein in Dengue patient samples obtained for further validation (second batch of sera).

FIG. 3B is a graph depicting levels of alpha-2 macroglobulin protein in Dengue patient samples obtained for further validation (second batch of sera).

FIG. 3C is a graph depicting levels of VEGF protein in Dengue patient samples obtained for further validation (second batch of sera).

FIG. 4A is a workflow diagram depicting screening of protein microarrays to identify novel autoantibodies in serum samples of Dengue patients.

FIG. 4B is a graph depicting the number of candidate biomarkers identified by cross-reactive antibodies that were significantly more prevalent in serum of Dengue patients than in healthy controls with Z-score values between 1 and 15.

FIG. 5 is a graph depicting the distribution according to cellular compartment of 67 autoantigens (Z-score above 3) identified by cross-reactive antibodies that were significantly more prevalent in Dengue patients than in healthy controls.

FIG. 6A is a graph depicting higher levels of VEGF in DF samples from Dengue Clinic (DC) with warning signs (WS) compared to those without warning signs (NWS).

FIG. 6B is a graph depicting levels of chymase-1 in DF samples from Dengue Clinic (DC) with warning signs (WS) compared to those without warning signs (NWS).

FIG. 6C is a graph depicting levels of alpha-2 macroglobulin in DF samples from Dengue Clinic (DC) with warning signs (WS) compared to those without warning signs (NWS).

FIG. 7A is a graph depicting levels of alpha-2 macroglobulin in severe Dengue samples compared to non-severe Dengue samples from Dengue Clinic (DC).

FIG. 7B is a graph depicting higher levels of VEGF in severe Dengue samples compared to non-severe Dengue samples from Dengue Clinic (DC).

FIG. 7C is a graph depicting levels of chymase-1 in severe Dengue samples compared to non-severe Dengue samples from Dengue Clinic (DC).

### DETAILED DESCRIPTION OF THE INVENTION

Dengue is a re-emerging arboviral disease with over half of the world's population living in areas of risk. Its etiological agent, the Dengue virus (DENV), causes a wide range of diseases, ranging from acute febrile Dengue fever (DF) to life-threatening Dengue hemorrhagic fever (DHF) and Dengue shock syndrome (DSS). No effective therapeutic option is currently available. Physicians can only rely on early recognition and prompt supportive treatment to lower the risk of patients developing DHF/DSS. However, there is a clear lack of relevant technology to accurately identify patients who will develop DHF/DSS. Dengue patients are hospitalized on a first-come-first-serve basis and this led to a great shortage of hospital beds and significant burden on the healthcare infrastructure during major Dengue epidemics. To address this issue, described herein is the identification of a panel of prognostic biomarkers that were identified using protoarray technology. The study described herein highlights a set of autoantigens to which Dengue-specific cross-reactive antibodies in Dengue patients' sera can bind more specifically and these novel autoantigens can be used as biomarkers in Dengue prognosis. The presence of identified autoantigens were also examined in Dengue patients' sera obtained from Early Dengue Infection and Outcome Study (EDEN study) and Adult Dengue Study (Dengue Clinic) at TTSH. VEGF, alpha-2 macroglobulin and chymase levels were significantly higher in DHF patients' sera. Statistical analysis revealed that alpha-2 macroglobulin and chymase can be used as prognostic biomarkers to differentiate DHF from DF and VEGF can be used to differentiate severe and non-severe forms of Dengue with the sensitivity and specificity in the range of 0.9. The application of the prognostic biomarkers identified herein will increase the ability of hospitals and doctors to prioritize and allocate valuable healthcare resources to the correct group of Dengue patients. Moreover this technology would bring about tremendous socioeconomic impacts in Dengue-affected countries by reducing hospitalization cost, improving the quality of hospital care, reducing patient suffering and saving lives.

### Methods of the Invention

The present invention provides, in one embodiment, a method of determining whether a subject in need thereof has a Dengue virus infection that is likely to develop into a life-threatening Dengue disease. The method of this embodiment comprises the steps of (a) detecting in a sample from the subject an expression level of at least two proteins selected from the group consisting of vascular endothelial growth factor protein (SEQ ID NO:1), chymase-1 protein (SEQ ID NO:2) and alpha-2 macroglobulin protein (SEQ ID NO:3); (b) comparing the expression level of each protein detected in (a) to a corresponding expression level for each protein in a control; and (c) determining that the patient has a Dengue virus infection that is likely to develop into a life-threatening Dengue disease when the expression level of each protein detected in the sample in (a) is significantly greater than the corresponding expression level for each protein in the control.

In various embodiments, the method comprises detecting in a sample an expression level of vascular endothelial growth factor protein (SEQ ID NO:1), chymase-1 protein (SEQ ID NO:2) and alpha-2 macroglobulin protein (SEQ ID NO:3), or any combination of two of these proteins, including an expression level of vascular endothelial growth factor protein (SEQ ID NO:1) and alpha-2 macroglobulin protein (SEQ ID NO:3) or a combination of chymase-1 protein (SEQ ID NO:2) and alpha-2 macroglobulin protein (SEQ ID NO:3).

As used herein, “vascular endothelial growth factor” or “VEGF” refers to a human vascular endothelial growth factor A (VEGFA) protein having the Accession Number BC065522.1 and the following amino acid sequence:

(SEQ ID NO: 1)
AAASRGQGPEPAPGGGVEGVGARGVALKLFVQLLGCSRFGGAVVR
AGEAEPSGAARSASSGREEPQPEEGEEEEEKEEERGPQWRLGARKPG
SWTGEAAVCADSAPAARAPQALARASGRGGRVARRGAEESGPPHSP
SRRGSASRAGPGRASETMNFLLSWVHWSLALLLYLHHAKWSQAAP
MAEGGGQNHHEVVKFMDVYQRSYCHPIETLVDIFQEYPDEIEYIFKP
SCVPLMRCGGCCNDEGLECVPTEESNITMQIMRIKPHQGQHIGEMSF
LQHNKCECRPKKDRARQEKCDKPRR.

“Chymase-1” or “chymase” refers to a human chymase-1 protein having the Accession Number EAW66007.1 and the amino acid sequence indicated below:

(SEQ ID NO: 2)
MLLLPLPLLLFLLCSRAEAGEIIGGTECKPHSRPYMAYLEIVTSNGPSK
FCGGFLIRRNFVLTAAHCAGRSITVTLGAHNITEEEDTWQKLEVIKQF
RHPKYNTSTLHHDIMLLKLKEKASLTLAVGTLPFPSQFNFVPPGRMC
RVAGWGRTGVLKPGSDTLQEVKLRLMDPQACSHFRDFDHNLQLCV
GNPRKTKSAFKGDSGGPLLCAGVAQGIVSYGRSDAKPPAVFTRISHY
RPWINQILQAN.

“Alpha-2 macroglobulin” or “macroglobulin” or “A2M” refers to a human alpha-2 macroglobulin protein having the Accession Number NM_{—}000014.3 and the amino acid sequence indicated below:

(SEQ ID NO: 3)
MGKNKLLHPSLVLLLLVLLPTDASVSGKPQYMVLVPSLLHTETTEKG
CVLLSYLNETVTVSASLESVRGNRSLFTDLEAENDVLHCVAFAVPKS
SSNEEVMFLTVQVKGPTQEFKKRTTVMVKNEDSLVFVQTDKSIYKP
GQTVKFRVVSMDENFHPLNELIPLVYIQDPKGNRIAQWQSFQLEGGL
KQFSFPLSSEPFQGSYKVVVQKKSGGRTEHPFTVEEFVLPKFEVQVTV
PKIITILEEEMNVSVCGLYTYGKPVPGHVTVSICRKYSDASDCHGEDS
QAFCEKFSGQLNSHGCFYQQVKTKVFQLKRKEYEMKLHTEAQIQEE
GTVVELTGRQSSEITRTITKLSFVKVDSHFRQGIPFFGQVRLVDGKGV
PIPNKVIFIRGNEANYYSNATTDEHGLVQFSINTTNVMGTSLTVRVNY
KDRSPCYGYQWVSEEHEEAHHTAYLVFSPSKSFVHLEPMSHELPCG
HTQTVQAHYILNGGTLLGLKKLSFYYLIMAKGGIVRTGTHGLLVKQE
DMKGHFSISIPVKSDIAPVARLLIYAVLPTGDVIGDSAKYDVENCLAN
KVDLSFSPSQSLPASHAHLRVTAAPQSVCALRAVDQSVLLMKPDAEL
SASSVYNLLPEKDLTGFPGPLNDQDDEDCINRHNVYINGITYTPVSST
NEKDMYSFLEDMGLKAFTNSKIRKPKMCPQLQQYEMHGPEGLRVGF
YESDVMGRGHARLVHVEEPHTETVRKYFPETWIWDLVVVNSAGVA
EVGVTVPDTITEWKAGAFCLSEDAGLGISSTASLRAFQPFFVELTMPY
SVIRGEAFTLKATVLNYLPKCIRVSVQLEASPAFLAVPVEKEQAPHCI
CANGRQTVSWAVTPKSLGNVNFTVSAEALESQELCGTEVPSVPEHG
RKDTVIKPLLVEPEGLEKETTFNSLLCPSGGEVSEELSLKLPPNVVEES
ARASVSVLGDILGSAMQNTQNLLQMPYGCGEQNMVLFAPNIYVLDY
LNETQQLTPEVKSKAIGYLNTGYQRQLNYKHYDGSYSTFGERYGRN
QGNTWLTAFVLKTFAQARAYIFIDEAHITQALIWLSQRQKDNGCFRS
SGSLLNNAIKGGVEDEVTLSAYITIALLEIPLTVTHPVVRNALFCLESA
WKTAQEGDHGSHVYTKALLAYAFALAGNQDKRKEVLKSLNEEAVK
KDNSVHWERPQKPKAPVGHFYEPQAPSAEVEMTSYVLLAYLTAQPA
PTSEDLTSATNIVKWITKQQNAQGGFSSTQDTVVALHALSKYGAATF
TRTGKAAQVTIQSSGTFSSKFQVDNNNRLLLQQVSLPELPGEYSMKV
TGEGCVYLQTSLKYNILPEKEEFPFALGVQTLPQTCDEPKAHTSFQIS
LSVSYTGSRSASNMAIVDVKMVSGFIPLKPTVKMLERSNHVSRTEVS
SNHVLIYLDKVSNQTLSLFFTVLQDVPVRDLKPAIVKVYDYYETDEF
AIAEYNAPCSKDLGNA.

The expression level of a vascular endothelial growth factor protein (SEQ ID NO:1), chymase-1 protein (SEQ ID NO:2) and/or alpha-2 macroglobulin protein (SEQ ID NO:3) is detected in a sample that is obtained from a subject in need thereof. A “subject in need thereof” encompasses vertebrates (e.g., mammals) who have a Dengue virus infection as well as vertebrates who are at risk for developing a Dengue virus infection. In a particular embodiment, the subject in need thereof is a human. A subject in need thereof can be a subject having one or more symptoms of a Dengue disease (e.g., a mild Dengue disease, a life-threatening Dengue disease) or a subject who is asymptomatic. Furthermore, the methods of the invention can be performed on a subject who is known to have a Dengue virus infection or a subject who is suspected of having a Dengue virus infection, but whose infection status is uncertain. Thus, the methods of the invention can be performed on a subject in need thereof before the onset of symptoms of a life-threatening Dengue disease or after the onset of symptoms of a life-threatening Dengue disease.

The sample obtained from the subject can be any suitable biological sample, including, but not limited to, whole blood, serum, plasma, urine, lymph fluid, cerebrospinal fluid, saliva, tissue biopsy, or a combination thereof. Preferably, the sample to be tested is a serum sample or plasma sample.

The expression level of a vascular endothelial growth factor protein (SEQ ID NO:1), chymase-1 protein (SEQ ID NO:2) or alpha-2 macroglobulin protein (SEQ ID NO:3) in a sample can be determined by detecting the protein itself in the sample from the subject, or by detecting antibodies (e.g., cross-reactive antibodies) to the protein in the sample from the subject.

Suitable techniques and assays for measuring the expression level of a protein (or antibodies thereto) in a sample (e.g., a biological sample) are well known in the art and include, for example, Western blotting techniques and various immunoassays. Preferably, an immunoassay is used to detect the expression level of vascular endothelial growth factor protein (SEQ ID NO:1), chymase-1 protein (SEQ ID NO:2) or alpha-2 macroglobulin protein (SEQ ID NO:3) in a sample from a subject. Immunoassays that are useful in the present invention include, but are not limited to, enzyme-linked immunosorbent assays (ELISAs), lateral flow immunoassays, radioimmunoassays, sandwich immunoassays, protein microarrays, magnetic immunoassays and surround optical fiber immunoassays.

In one embodiment, an ELISA is used to detect the expression level of vascular endothelial growth factor protein (SEQ ID NO:1), chymase-1 protein (SEQ ID NO:2) or alpha-2 macroglobulin protein (SEQ ID NO:3) in a sample from a subject. In another embodiment, a lateral flow immunoassay is used to detect the expression level of vascular endothelial growth factor protein (SEQ ID NO:1), chymase-1 protein (SEQ ID NO:2) or alpha-2 macroglobulin protein (SEQ ID NO:3) in a sample from a subject.

In yet another embodiment, a protein microarray is used to detect antibodies to a vascular endothelial growth factor protein (SEQ ID NO:1), chymase-1 protein (SEQ ID NO:2) or alpha-2 macroglobulin protein (SEQ ID NO:3) in a sample from a subject. Useful protein microarrays are available commercially and include, for example, ProtoArray® Human Protein Microarrays (Life Technologies, Grand Island, N.Y.).

The methods of the invention further comprise the step of comparing the expression level of each protein detected in the sample from the subject to a corresponding expression level for each protein in a control. As used herein, a “corresponding expression level” can be an actual expression level of a protein in a sample obtained from a control (e.g., a healthy human subject) or a standard reference protein level that is indicative of the typical or average level of the protein in a healthy human subject.

The methods of the invention further comprise the step of determining that the patient has a Dengue virus infection that is likely to develop into a life-threatening Dengue disease when the expression level of each protein detected in the sample from the subject is significantly greater than the corresponding expression level for each protein in the control. In one embodiment, the expression level of vascular endothelial growth factor protein (SEQ ID NO:1), chymase-1 protein (SEQ ID NO:2) or alpha-2 macroglobulin protein (SEQ ID NO:3) in the sample from the subject is significantly greater than the corresponding expression level for the relevant protein in the control when there is a statistically greater expression level of the protein in the sample compared to the control. In a particular embodiment, the statistically greater expression level in the sample compared to the control is at a level of about 5% significance (95% confidence interval). Suitable statistical tests for evaluating whether a protein expression level in a sample is significantly greater than a protein level in a control are well known in the art and include, for example, the statistical tests employed in the Examples described herein.

In another embodiment, the method of determining whether a subject in need thereof has a Dengue virus infection that is likely to develop into a life-threatening Dengue disease further comprises the step of administering a treatment regimen for a life-threatening Dengue disease to the subject upon determining that the patient has a Dengue virus infection that is likely to develop into a life-threatening Dengue disease.

As used herein, a “life-threatening Dengue disease” includes, for example, Dengue hemorrhagic fever (DHF), Dengue shock syndrome (DSS), severe Dengue and Dengue with warning signs. DHF is characterized by increased vascular permeability, thrombocytopenia and hemorrhagic manifestations. Common hemorrhagic manifestations include skin hemorrhages such as petechiae, purpuric lesions and ecchymoses. Epistaxis, bleeding gums, gastro-intestinal hemorrhage and hematuria occur less frequently. DSS occurs when fluid leakage into the interstitial spaces results in shock, which without appropriate treatment may lead to death. Both DHF and DSS can be diagnosed clinically according to World Health Organization (WHO) criteria published in “Dengue haemorrhagic fever: Diagnosis, treatment, prevention and control”, 2^{nd} Ed., Geneva (1997), the relevant contents of which are incorporated herein by reference.

Severe Dengue and Dengue with warnings signs can be classified according to WHO criteria published in “Dengue: Guidelines for Diagnosis, Treatment, Prevention and Control”, Geneva (2009), the relevant contents of which are incorporated herein by reference. According to the WHO criteria, characteristics of severe Dengue include severe plasma leakage, severe bleeding/haemorrhage, or severe organ impairment/failure, while characteristics of Dengue with warning signs include abdominal pain or tenderness, persistent vomiting, fluid accumulation, mucosal bleeding, lethargy, restlessness, liver enlargement, increasing haematocrit with rapid decrease in platelet count.

Treatment regimens for a life-threatening Dengue disease are described in the WHO publications “Dengue haemorrhagic fever: Diagnosis, treatment, prevention and control”, 2^{nd} Ed., Geneva (1997) and “Dengue: Guidelines for Diagnosis, Treatment, Prevention and Control”, Geneva (2009). For example, a treatment regimen for a life-threatening Dengue disease can include one or more of the following: hospitalization, administration of antipyretics (e.g., paracetamol), intake of fluid (e.g., water, electrolyte replacement solution, fruit juice) by mouth, platelet transfusion, sedative therapy, and oxygen therapy. A skilled physician can readily determine and administer an appropriate treatment regimen, including suitable dosages of therapeutic agents, based on relevant patient characteristics (e.g., age, weight, severity of symptoms, existing or prior medical conditions).

In a further embodiment, the invention relates to a method of selecting a treatment regimen for a subject in need thereof who has been infected with a Dengue virus. The method of selecting comprises (a) detecting in a sample from the subject an expression level of at least two proteins selected from the group consisting of vascular endothelial growth factor protein (SEQ ID NO:1), chymase-1 protein (SEQ ID NO:2), and alpha-2 macroglobulin protein (SEQ ID NO:3); (b) comparing the expression level of each protein detected in (a) to a corresponding expression level for each protein in a control; and (c) selecting either: (1) a regimen for treating a life-threatening Dengue disease when the expression level of each protein detected in the sample in (a) is significantly greater than the corresponding expression level for each protein in the control; or (2) a regimen for treating a mild Dengue disease when the expression level of each protein detected in the sample in (a) is not significantly greater than the corresponding expression level for each protein in the control.

In a particular embodiment, the method of selecting a treatment regimen further comprises administering the selected regimen to the subject. The selected regimen can be administered to the subject before the onset of symptoms of a life-threatening Dengue disease or after the onset of symptoms of a life-threatening Dengue disease.

In the method of selecting, the detecting and comparing steps are performed as described herein above. A regimen for treating a life-threatening Dengue disease is selected for the subject when the expression level of each protein detected in the sample is significantly greater than the corresponding expression level for each protein in the control. A suitable regimen for treating a life-threatening Dengue disease can be readily determined by a skilled physician and can include, for example, one or more of the treatments for a life-threatening Dengue disease disclosed above.

Alternatively, when the expression level of the protein detected in the sample from the subject is not significantly greater than the corresponding expression level for the protein in the control, a regimen for treating a mild Dengue disease is selected. As used herein, a “mild Dengue disease” includes, for example, Dengue fever (DF), as defined by WHO criteria published in “Dengue haemorrhagic fever: Diagnosis, treatment, prevention and control”, 2^{nd} Ed., Geneva (1997), and Dengue without warning signs, as defined by WHO criteria published in “Dengue: Guidelines for Diagnosis, Treatment, Prevention and Control”, Geneva (2009). DF is characterized by such symptoms as undifferentiated febrile disease, mild febrile syndrome, high fever with abrupt onset, severe headache, aches and pains, nausea, vomiting and rash. Dengue without warnings signs is characterized by fever and at least 2 symptoms selected from nausea/vomiting, rash, aches and pains, positive tourniquest test, and leukopenia without exhibiting any of the symptoms of Dengue with warning signs.

A treatment regimen (e.g., outpatient treatment regimen) for a mild Dengue disease can include, for example, increasing fluid intake and monitoring (e.g., daily monitoring) of the patient by healthcare providers for signs of disease progression and/or warning signs. In some cases, no treatment will be administered to a subject having a mild Dengue disease and the subject will be sent home without further monitoring.

### Kits of the Invention

The present invention also provides a prognostic kit for distinguishing a subject who is likely to develop a mild Dengue disease from a subject who is likely to develop a life-threatening Dengue disease. The kit comprises at least two of: (a) one or more reagents for detecting an expression level of: (1) a vascular endothelial growth factor protein, (2) an antibody to a vascular endothelial growth factor protein, or (3) a combination thereof; (b) one or more reagents for detecting an expression level of: (1) a chymase-1 protein, (2) an antibody to a chymase-1 protein, or (3) a combination thereof; or (c) one or more reagents for detecting an expression level of: (1) an alpha-2 macroglobulin protein, (2) an antibody to an alpha-2 macroglobulin protein, or (3) a combination thereof.

In one embodiment, the kit comprises reagents (e.g., antibodies, aptamers) for detecting the expression level of vascular endothelial growth factor protein (SEQ ID NO:1), chymase-1 protein (SEQ ID NO:2), and alpha-2 macroglobulin protein (SEQ ID NO:3). In a particular embodiment, the reagents are antibodies. As used herein, the term “antibody” is intended to encompass both whole antibodies and antibody fragments (e.g., antigen-binding fragments of antibodies, for example, Fv, Fc, Fd, Fab, Fab′, F(ab′), and dAb fragments). “Antibody” refers to both polyclonal and monoclonal antibodies and includes naturally-occurring and engineered antibodies. Thus, the term “antibody” includes, for example, human, chimeric, humanized, primatized, veneered, single chain, and domain antibodies (dAbs). (See e.g., Harlow et al., Antibodies A Laboratory Manual, Cold Spring Harbor Laboratory, 1988). Preferably, the antibodies in the kit are immobilized on a solid support. Suitable solid supports for immobilization of proteins, including antibodies, are well known in the art.

In another embodiment, the kit comprises reagents for detecting the expression level of antibodies to a vascular endothelial growth factor protein (SEQ ID NO:1), antibodies to a chymase-1 protein (SEQ ID NO:2), and antibodies to an alpha-2 macroglobulin protein (SEQ ID NO:3). Preferably, the reagents are immobilized vascular endothelial growth factor (SEQ ID NO:1), chymase-1 (SEQ ID NO:2), and alpha-2 macroglobulin (SEQ ID NO:3) proteins. The immobilized proteins can be full-length proteins, partial proteins or peptides. In one embodiment, the protein reagents in the kits of the invention are provided on a protein microarray.

Preferably, the reagents (e.g., antibodies, proteins, peptides) in the kits of the invention comprise one or more detectable labels. Labels suitable for use according to the present invention are known in the art and generally include any molecule that, by its chemical nature, and whether by direct or indirect means, provides an identifiable signal allowing detection of the probe. Thus, for example, reagents may be labeled in a conventional manner, such as with specific reporter molecules, fluorophores, radioactive materials, or enzymes (e.g., peroxidases, phosphatases).

Detectable labels suitable for attachment to reagents can be indirect labels or direct labels. Exemplary indirect labels include, e.g., haptens, biotin, or other specifically bindable ligands. For indirect labels, the ligand-binding partner typically has a direct label, or, alternatively, is also labeled indirectly. Examples of indirect labels that are haptens include dinitrophenol (DNP), digoxigenin, biotin, and various fluorophores or dyes (e.g., fluorescein, DY490, DY590, Alexa 405/Cascade blue, Alexa 488, Bodiby FL, Dansyl, Oregon Green, Lucifer Yellow, Tetramethylrhodamine/Rhodamine Red, and Texas Red). As an indirect label, a hapten is typically detected using an anti-hapten antibody as the ligand-binding partner. However, a hapten can also be detected using an alternative ligand-binding partner (e.g., in the case of biotin, anti-biotin antibodies or streptavidin, for example, can be used as the ligand-binding partner). Further, in certain embodiments, a hapten can also be detected directly (e.g., in the case of fluorescein, an anti-fluorescein antibody or direct detection of fluorescence can be used).

Exemplary “direct labels” include, but are not limited to, fluorophores (e.g., fluorescein, rhodamine, Texas Red, phycoerythrin, Cy3, Cy5, DY fluors (Dyomics GmbH, Jena, Germany) Alexa 532, Alexa 546, Alexa 568, or Alexa 594). Other direct labels can include, for example, radionuclides (e.g., 3H, 35S, 32P, 125I, and 14C), enzymes such as, e.g., alkaline phosphatase, horseradish peroxidase, or β-galactosidase, chromophores (e.g., phycobiliproteins), luminescers (e.g., chemiluminescers and bioluminescers), and lanthanide chelates (e.g., complexes of Eu3+ or Tb3+). In the case of fluorescent labels, fluorophores are not to be limited to single species organic molecules, but include inorganic molecules, multi-molecular mixtures of organic and/or inorganic molecules, crystals, heteropolymers, and the like. For example, CdSe-CdS core-shell nanocrystals enclosed in a silica shell can be easily derivatized for coupling to a biological molecule (Bruchez et al., Science, 281:2013-2016, 1998). Similarly, highly fluorescent quantum dots (zinc sulfide-capped cadmium selenide) have been covalently coupled to biomolecules for use in ultrasensitive biological detection (Warren and Nie, Science, 281: 2016-2018, 1998).

In another embodiment, the kits of the invention further comprise components for performing an immunoassay. Preferably, the immunoassay is a rapid test assay which can be completed in less than 45 minutes.

In one embodiment, the kit comprises reagents and components for performing an ELISA. Reagents and components for performing an ELISA are well known in the art and include, for example, an antibody-coated plate (e.g., microwell plate), a detection antibody, standards, detection reagents, diluents, wash buffer, stop solution, and plate sealers.

In another embodiment, the kit comprises reagents and components for performing a lateral flow assay. For example, the kit can include one or more test strips for a lateral flow assay. Typically, a test strip comprises a sample pad to which the test sample is applied, a conjugate or reagent pad containing detectably labeled antibodies specific to the target, a reaction membrane (e.g., nitrocellulose, cellulose acetate) to which antibodies are immobilized, and a wick or waste reservoir to draw the sample across the reaction membrane and collect it.

The kits of the invention can also include, for example, instructions for the kit user.

A description of example embodiments of the invention follows.

### Example 1

### Identification of Host Biomarkers Differentiating Dengue Fever (DF) and Dengue Hemorrhagic Fever (DHF)

Dengue virus (DENV) infection is a re-emerging infectious disease that accounts for a hundred million cases annually. However, there are no vaccine and effective therapeutic options currently available. Early recognition and prompt supportive treatment can help to lower the risk of developing severe disease complications such as Dengue Hemorrhagic Fever/Dengue Shock Syndrome (DHF/DSS). Hence there is an urgent need to identify the biomarkers linked with DHF/DSS. Thus, this study aimed to identify potential biomarkers that are linked with varying degrees of disease severity and capable of differentiating Dengue Hemorrhagic Fever/Dengue Shock Syndrome (DHF/DSS) and Dengue fever (DF).

Currently, diagnosis is based on the virus factors (NS1) or detecting the IgG and IgM antibodies.

To identify a good matrix of predictive biomarkers from the host perspective to differentiate DF and DHF cases ProtoArray® Human Protein Microarrays that consisted of 9000 host proteins were utilized. This array provides absolute identification of auto-antibodies/proteins (biomarkers) present in the patient serum/plasma. The identity of every antigen printed on the array is known and addressable. The array includes specific controls to monitor assay quality and normalize data and this leads to the accurate detection of biomarkers.

The sera collected from Dengue patients with varying degrees of disease severity were used to probe the protoarray chips. The samples were collected at three different time points from each of patients DKI, DK2 and DK3 (DKI: 1-3 days after the onset of fever; DK2: 4-7 days after the onset of fever; DK3: 21-28 days after the onset of fever) in EDEN study (retrospective). 46 different proteins involved in signal transduction, membrane permeability, intracellular trafficking, enzymatic activity, transcription, muscle functions, immune response and apoptosis were found to be stimulated during Dengue infections.

Three proteins were selected for this study (using ELISA) because their levels were significantly higher in DHF sera and their functions are closely related to vascular permeability and blood coagulation. They are:
Vascular endothelial growth factor (VEGF): Highly specific mitogenic activity for endothelial cells and angiogenic activity.
Chymase-1: Functions in the degradation of the extracellular matrix, regulation of submucosal gland secretion, and generation of vasoactive peptides.
Alpha 2 macroglobulin: Inactivates an enormous variety of proteinases, inhibitor of fibrinolysis, acts as a carrier protein by binding to numerous growth factors and cytokines, such as platelet-derived growth factor, basic fibroblast growth factor, TGF-β, insulin, and IL-1β.

Investigating these proteins would help us to identify valuable biomarkers to predict patients at risk of developing DHF/DSS. Sera and plasma samples obtained from EDEN study and DC clinic were used for downstream analyses.

Retrospective Study

### VEGF

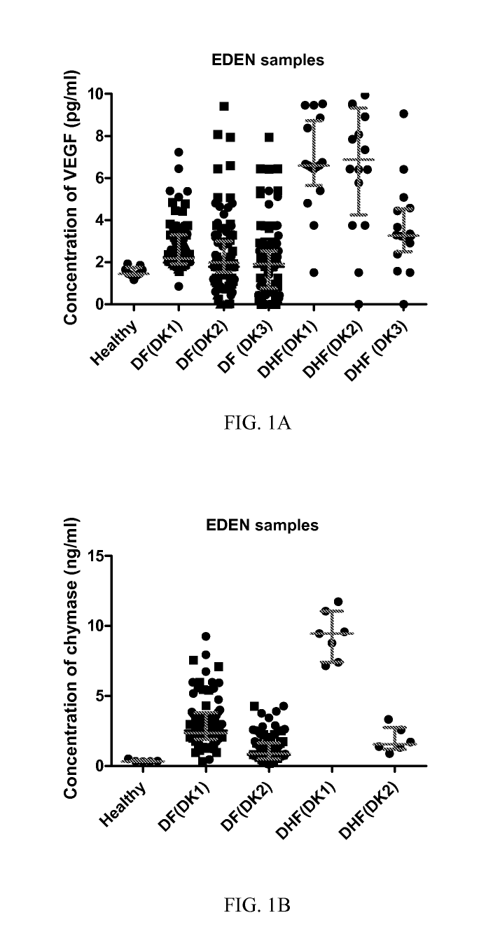

Among the 124 serum samples selected from EDEN, 108 DF serum samples and 16 DHF serum samples were used. VEGF was significantly increased (p<0.05) in DHF samples compared to that of DF samples (FIG. 1A). The increase was predominantly higher at the early stages of the disease (DK1 and DK2) and decreased subsequently (DK3). Although some DF samples showed a moderate increase, the increased levels in DHF patients were found to be significantly higher.

### Chymase

Chymase levels were significantly increased (p<0.05) in DK1 samples of DHF sera compared to that of DF sera samples (FIG. 1B). The increase was predominant at the early stage of the disease and decreased subsequently in DK3 samples.

In summary, the data obtained from retrospective study highlighted that VEGF and chymase could be used as biomarkers to differentiate DF and DHF.

### Prospective Study

Following the promising results obtained from the retrospective study, the study was extended with the samples from a prospective study. Macroglobulin was included into the list of biomarkers for analysis. The results are presented with DHF, severe Dengue, or warning signs as endpoints:

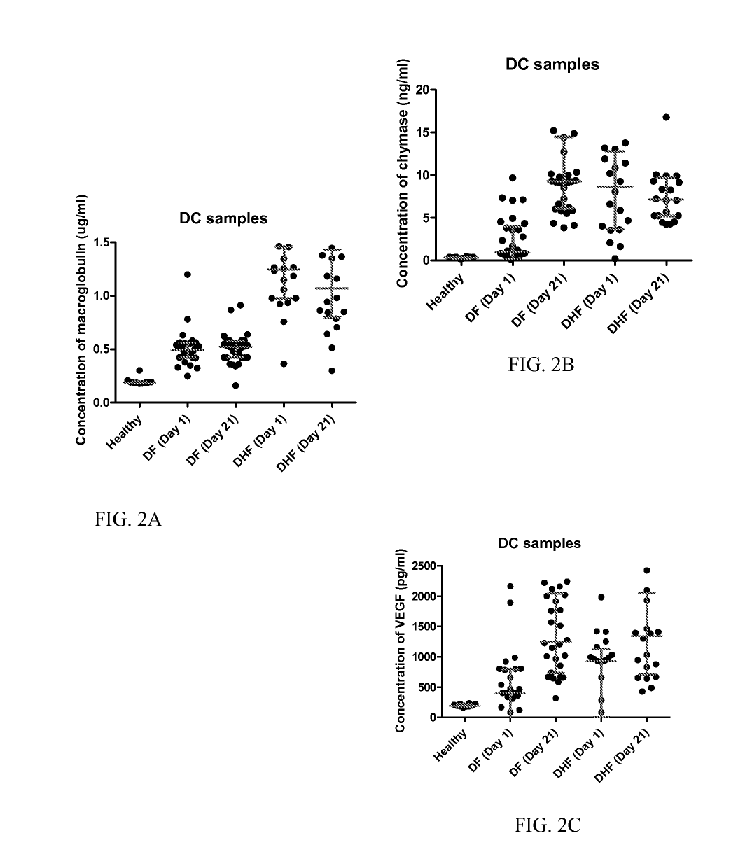

First Batch of Sera
(i) DHF/Severe prediction
Results shown in FIGS. 2A-C.

TABLE 1
The statistics generated from the 1st
batch of patient sera from Dengue Clinic

| Model | AUC | Sensitivity | Specificity |
|---|---|---|---|
| Chymase | 0.758 | 0.55 | 0.967 |
| VEGF | 0.717 | 0.60 | 0.830 |
| α macroglobulin | 0.942 | 0.95 | 0.933 |
| Chymase + macro | 0.933 | 0.90 | 0.967 |

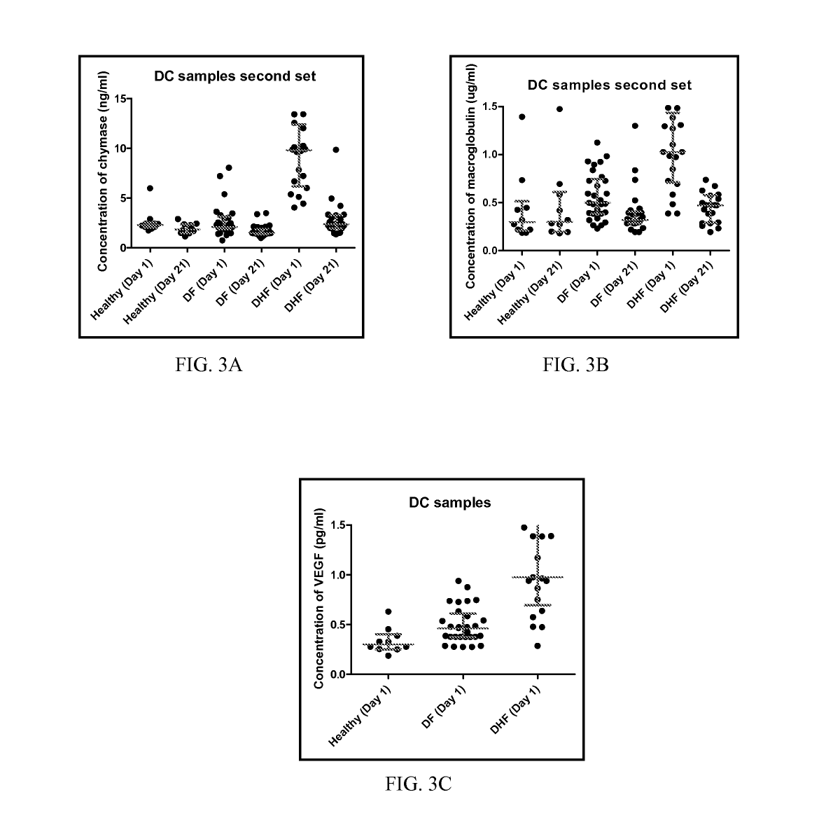

Second Batch of Sera (for Further Validation)
Results shown in FIGS. 3A-C.

TABLE 2
The statistics generated from the 2nd
batch of patient sera from Prospective Study

| Model | AUC | Sensitivity | Specificity |
|---|---|---|---|
| Chymase (cut off 5.66) | 0.94 | 0.81 | 0.9 |
| Cut off: 3.83 | | 1 | 0.86 |
| VEGF | 0.89 | 0.76 | 0.93 |
| α macroglobulin | 0.84 | 0.67 | 0.93 |
| Chymase + macro + VEGF | 0.96 | 0.9 | 0.97 |

TABLE 3
Combined results - 1st and 2nd batches
of patient sera from Prospective Study

| Model | AUC | Sensitivity | Specificity |
|---|---|---|---|
| Chymase | 0.89 | 0.9 | 0.78 |
| VEGF | 0.63 | 0.76 | 0.6 |
| α macroglobulin | 0.9 | 0.756 | 0.95 |
| Chymase + marco | 0.97 | 0.93 | 0.9 |

Overall, this study pinpointed that macroglobulin in combination with chymase and VEGF served as good predictive biomarkers to differentiate DF and DHF cases giving the sensitivity and specificity of greater than 0.9.

### Significance:

Identification of credible biomarkers related to disease outcome can assist the clinicians to recommend appropriate supportive care/treatment at the early stages. Moreover, the existence of credible predictive biomarkers to differentiate between DF and DHF would lead to saving of hospitalization cost.

The diagnostic kits can be developed utilizing these panel of three biomarkers which will be of commercial value. The unique thing about the biomarkers identified herein is that, as a panel of biomarkers, they can differentiate between DF and DHF, between severe and non-severe dengue and between DF with and without warning signs.

Based on the findings described herein, a variety of tests for use at point of care level (e.g., a dip test) can be used.

### Example 2

### Identification of Host Biomarkers Differentiating Life-Threatening and Mild Forms of Dengue Fever

In order to identify reliable Dengue prognostic biomarkers, it is essential to understand virus morphogenesis and the molecular basis of pathogenesis. Viruses, in general, attempt to subvert host cell processes to increase the efficiency of virus infection and likewise the cell employs a number of responses to generate an anti-viral state. The pathogenesis of severe Dengue disease is also a complex, multifactorial and coordinated process involving the interaction between cellular and viral networks. Several theories such as virus virulence, antibody-dependent enhancement of infection and abnormal host immune responses (cytokines and chemokines production, complement activation and apoptotic cell death) have been proposed to explain the pathogenesis of the DHF/DSS (5-10). In addition, autoimmunity was shown to play a role in inducing DHF/DSS. Dengue-induced cross-reactive autoantibodies could interfere with platelet, endothelial cells or coagulation activity because of molecular mimicry between Dengue virus proteins and human proteins (10, 11). Nevertheless, the main mechanism underlying the development of DHF/DSS remains unknown. Understanding how cellular proteins interact with Dengue virus, as well as the significance of Dengue-induced immune responses, is crucial to understand the pathogenesis of DHF/DSS.

The previously reported autoantibodies were targeted towards platelet, endothelial cells or coagulation factors (10, 11). Whether Dengue-induced cross-reactive autoantibodies are directed specifically towards platelet, endothelial cells, coagulation factors or a wide variety of other cellular factors involved in modulating various cellular responses and contributing to virus pathogenesis were explored. ProtoArray Human Protein Microarrays (Invitrogen) were used to perform high-throughput screening of novel autoantibodies in Dengue disease. This study demonstrated the presence of several cross-reactive antibodies or autoantibodies in Dengue patients' sera. More interestingly, a group of Dengue-specific cross-reactive antibodies were found at significantly higher levels in the serum of DHF patients.

Some of the results discussed below include data and results described herein in Example 1.

### Subjects and Methods

### Ethics Statement

Informed consent was obtained and all procedures were carried out under an approved protocol from the National University Institutional Review Board (NUS-IRB number 06-196) and NHG Review Board (DSRB Ref: B/05/013 and DSRB Ref: E/09/432).

### Subjects and Materials

Blood samples were collected into vacutainer tubes from suspected Dengue patients and incubated in an upright position at room temperature for 45 min. The supernatant (serum) was carefully aspirated and centrifuged if turbid. The serum samples were aliquoted into cryovials and stored at −80° C. until use. Diagnosis of Dengue virus infection and serotyping was performed at Life Sciences Institute (NUS, Singapore), Environmental Health Institute (Singapore), Genomic Institute of Singapore (Singapore) and Tan Tock Seng Hospital (TTSH, Singapore) using anti-Dengue enzyme-linked immunosorbent assay, serotype-specific reverse transcription-polymerase chain reaction (RT-PCR) and plaque reduction neutralization assay.

Dengue patients were recruited in 2 cohorts. The first cohort of patients and controls consisted of 130 Dengue patients, 10 Dengue-negative (febrile) patients and 10 healthy controls. These subjects were enrolled for the Early Dengue Infection and Outcome Study (EDEN study) and were retrospectively included in this study following Institutional Review Board's approval. The enrolled Dengue cases consisted of all 4 serotypes of Dengue (D1 to D4). Out of 130 Dengue cases, 16 were DHF cases and the rest were DF cases. DF and DHF patients were characterized based on WHO 1997 classification. The sera samples were collected at three different time points from each of patients DKI, DK2 and DK3 (DKI: 1-3 days; DK2: 4-7 days; DK3: 3-4 weeks). The patients with fever greater than 38° C. at the first visit, with a mean duration of 43 hours (range 14-72) from the onset of the fever was selected for this study. The mean age of DF patients was 38.9 years (range 18-66) and that of DHF patients was 45.75 years (range (24-77). Dengue negative patients were enrolled with the same criteria (fever with no respiratory infection symptoms), but were RT-PCR negative for Dengue. The patient characteristics were summarized in Table 4.

The second cohort of patients and controls consisted of 100 Dengue patients, 10 Dengue-negative (febrile) patients and 10 healthy controls. These subjects were prospectively recruited from Adult Dengue Study (Dengue Clinic) at TTSH. Out of 100 Dengue cases, 41 subjects were diagnosed as DHF cases and the rest were DF cases. The enrolled Dengue cases consisted of all 4 serotypes of Dengue (D1 to D4). DF and DHF patients were characterized based on WHO 1997 classification. The sera samples were collected at two time points from each patient (acute 1-3 days; Convalescent 21 days). The mean fever for DF patients was 37.5° C. (range 36-39.3) and that of DHF patients was 37.61° C. (range 36.5-39). The mean age of DF patients was 33.84 years (range 20-63) and that of DHF patients was 41.95 years (range (21-74). The patient characteristics are summarized in Table 5.

### Protein Microarray to Screen for Cross-Reactive Autoantibodies

Protoarray® Human Protein Microarrays (v5) for Immune Response Biomarker Profiling (Invitrogen) consisted of over 9,000 unique human proteins individually purified and arrayed under native conditions to maximize functionality. This array included control ProtoArray® Human Protein Microarray proteins in each sub-array for normalization purposes. The experiment was performed following manufacturer's instructions. In brief, all the manipulations were performed at 4° C. Protoarray slides were treated with blocking buffer (PBS, pH 7.4, containing 1% BSA and 0.1% Tween 20). Serum samples were diluted (1:500) in a probe buffer (PBS, pH 7.4, 5 mM MgCl₂, 0.05% Triton X-100, 1% glycerol, and 1% BSA) and used to probe the protoarray slides. The arrays were washed three times with probe buffer and Alexa Fluor 647-conjugated anti-human IgG antibody (1 μg/ml buffer) was added. The arrays were washed and dried by centrifugation at 200 g for 2 min at room temperature. Arrays were then scanned using GenePix 4000B fluorescent microarray scanner (Molecular Devices Corporation, USA) within 24 h. Raw pixel counts were generated by scanning arrays at 635 nm using a photomultiplier tube gain setting of 500 and a power setting of 100%.

### Data Acquisition and Analysis

GenePix Pro 6.0 software (Molecular Devices Corporation, USA) was used to align the scanned image to the lot-specific ‘GAL’ file downloaded from ProtoArray Central (Invitrogen) and to determine the pixel intensities for each spot on the array. Acquired data were analyzed using ProtoArray Prospector software (Invitrogen) in Immune Response Profiling mode. This software performs background subtraction, normalization of the signals, and analysis of the differences between two groups of patients. Data were analyzed by calculation of Z score, Chebyshev's inequality precision value (CIP) and the co-efficient of variation (CV). A positive spot is defined by a Z score of >3, a CIP value of <0.05 and a CV of <0.5. The functions and subcellular locations of the autoantigens were analyzed via GeneCards (http://www.genecards.org), UniProt (http://www.uniprot.org) and The Gene Ontology (http://www.geneontology.org). Protein categorization was then performed based on the obtained information.

### Detection of Cross-Reactive Autoantigens

The authenticity of the identified cross-reactive autoantigens was validated by performing an enzyme linked immunosorbent assay (ELISA)-based binding assay using alpha 2 Macroglobulin Human ELISA Kit (Abcam), Human VEGF ELISA kit (Thermo Scientific) and Chymase 1, Mast Cell (CMA1) ELISA Kit (Antibodies online) following manufacturer's instructions. The protocol for each kit is briefly described below.

### Alpha-2 Macroglobulin Human ELISA Kit (Abcam)

After bringing all the reagents, samples and standards to room temperature, 25 μl standard or sample was added to each well and immediately 25 μl prepared biotin antibody was added to each well. The plate was then incubated for 2 hours at room temperature. This was followed by washing five times with wash buffer. Then, 50 μl of Streptavidin-Peroxidase Conjugate was added and incubated for 30 minutes at room temperature. Following five washes with wash buffer, 50 μl of Chromogen Substrate was added to each well and incubated for 30 minutes and 50 μl Stop Solution was added to each well. The absorbance was immediately measured on an ELISA plate reader set at 450 nm.

### Chymase 1, Mast Cell (CMA1) ELISA Kit (Antibodies Online)

After bringing all the reagents, samples and standards to room temperature, 100 μl of standard or sample was added to each well and incubated for 2 hours at 37° C. Then 100 μl of prepared Detection Reagent A was added to each well and incubated for 1 hour at 37° C. Following washing three times with wash buffer, 100 μl of prepared Detection Reagent B was added to each well. The plate was incubated for 30 minutes at 37° C. before washing five times with wash buffer. Then 90 μl of Substrate Solution was added and the wells were incubates for 30 min at 37° C. before adding 50 μl of Stop Solution. The absorbance was immediately measured on an ELISA plate reader set at 450 nm.

### Human VEGF ELISA Kit (Thermo Scientific)

After bringing all the reagents, samples and standards to room temperature, 50 μl of Sample Diluent was added to each well followed by the addition of 50 μl of standard or sample to each well. The plate was covered with adhesive plate sealer and incubated for 2 hours at room temperature, 20-25° C. Following three washes with wash buffer, 100 μl of Biotinylated Antibody Reagent was added to each well and incubated for 1 hour at room temperature, 20-25° C. This was followed by three washes with wash buffer. Then 100 μl of Streptavidin-HRP Reagent was added to each well and incubated for 30 minutes at room temperature, 20-25° C. Following three washes with wash buffer, 100 μl of TMB Substrate Solution was added into each well and incubated for 30 minutes. After 30 minutes, the reaction was stopped by adding 100 μl of Stop Solution to each well. The absorbance was immediately measured on an ELISA plate reader set at 450 nm.

### Statistical Analysis

Statistical analysis for the microarray studies was described above (under Data Acquistion and Analysis). Results of ELISA experiments were evaluated by means of Student's T-test. Continuous variables were summarized using mean and standard deviation/range. All statistical tests were set at a 5% level of significance. Confidence intervals were 95% and two-sided. Statistical analyses will be performed using Prism software.

### Results

### Profiling Autoimmune Signatures in Dengue Patients

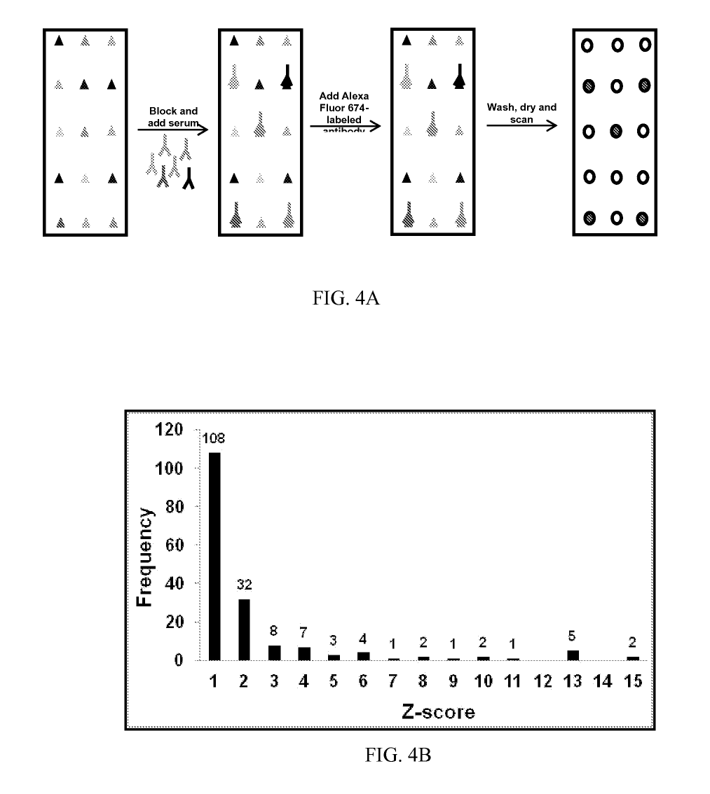

Protoarray® Human Protein Microarrays for Immune Response Biomarker Profiling was used to identify novel autoantibodies in the serum samples of Dengue patients. The DK1 samples from 14 Dengue patients and 10 healthy controls (EDEN study) were used. The workflow of the experiment was illustrated in FIG. 4A. ProtoArray Prospector software was used to reveal differences between Dengue patients and healthy subjects. Antibodies to 196 antigens were detected in Dengue patients than in healthy controls and these antibodies showed a Z-score value of above 1 (FIG. 4B). The selection stringency was increased by setting the cut-off at the Z-score value above 3 and this eliminated the potential false positives. Z-score indicates how far and in what direction the sample's value deviates from the distribution's mean. After filtering some of the proteins without known functions, 67 exclusive autoantibodies were identified in the serum samples of Dengue patients.

### Categorization of Candidate Autoantigens Against Candidate Cross-Reactive Antibodies

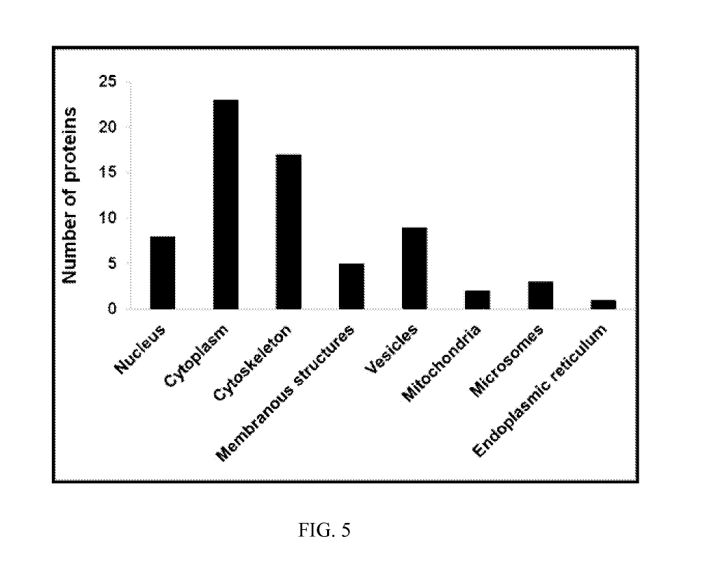

The screening experiment identified 67 antigens (Z-score above 3) to which antibodies were significantly more prevalent in Dengue patients than in controls. The function of these candidate autoantigens was examined using GeneCards, UniProt and The Gene Ontology software and categorized based on their functional relevance and disease pathogenesis. As shown in FIG. 5, the identified autoantigens showed a wide range of cellular distribution. The majority of these proteins were known to modulate membrane permeability, intracellular trafficking, signal transduction pathways, enzymatic activity, muscular functions, immune responses, apoptosis and degradation pathways.

### Identification of Severe Dengue Prognostic Biomarkers

In order to examine whether the identified candidate autoantigens could differentiate DHF/DSS from DF cases, the ELISA results obtained from 30 autoantigens were re-analyzed by grouping Dengue patients into two categories (DF and DHF). Interestingly, cross-reactive antibodies to 7 autoantigens were significantly more prevalent in the serum samples of DHF patients compared to that of DF patients. The function of the novel autoantigens to which autoantibodies are present in DHF patients' sera were categorized depending on their function. The majority of the proteins against candidate cross-reactive antibodies are related to signal transduction, intracellular trafficking, enzymatic activity, membrane permeability and muscular functions. A group of proteins that are present significantly higher in DHF patients have also been identified.

### Validation of Novel Autoantigens by ELISA

Three proteins were selected because their levels were significantly higher in DHF sera and their functions are closely related to vascular permeability and blood coagulation. They are vascular endothelial growth factor-A (VEGF), alpha-2 macroglobulin, and chymase.

Vascular endothelial growth factor (VEGF—also known as vascular permeability factor) possesses highly specific mitogenic activity for endothelial cells and angiogenic activity. Chymase-1 is a chymotryptic serine proteinase and is expressed in mast cells and thought to function in the degradation of the extracellular matrix, the regulation of submucosal gland secretion and the generation of vasoactive peptides. In the heart and blood vessels, it is largely responsible for converting angiotensin I to the vasoactive peptide angiotensin II. Angiotensin II has been implicated in blood pressure control and in the pathogenesis of hypertension, cardiac hypertrophy, and heart failure. Alpha 2 macroglobulin acts as an antiprotease and inactivates an enormous variety of proteinases. It functions as an inhibitor of fibrinolysis and coagulation by inhibiting plasmin and kallikrein and thrombin. It also acts as a carrier protein because it also binds to numerous growth factors and cytokines, such as platelet-derived growth factor, basic fibroblast growth factor, TGF-β, insulin, and IL-1β. Investigation of these proteins identified valuable biomarkers to predict patients at risk of developing DHF/DSS. Sera and plasma samples obtained from EDEN study and DC clinic were used for downstream analyses.

Data Obtained from EDEN Study VEGF

Among the 124 serum samples selected from EDEN, 108 DF serum samples and 16 DHF serum samples were used. As shown in FIG. 1A, VEGF was significantly increased (P<0.05) in DHF samples compared to that of DF samples. The increase was predominantly higher at the early stages of the disease (DK1 and DK2) and decreased subsequently (DK3). Although some DF samples showed a moderate increase, the increased levels in DHF patients were found to be significantly higher.

Some DF samples reached the value similar to that of DHF samples; this was likely due to the following reasons: These DF cases could have been clinically diagnosed as DF but physiologically experiencing DHF symptoms, or, in other words, they could have been borderline cases between DF and DHF. Nevertheless, these results confirmed that VEGF can serve as a biomarker to differentiate DF and DHF (although the specificity was low).

### Chymase

Chymase levels were significantly increased (P<0.05) in DK1 samples of DHF sera (FIG. 1B) compared to that of DF sera samples. The increase was predominant at the early stage of the disease and decreased subsequently in DK3 samples.

### Conclusion

In summary, the data obtained from EDEN study highlighted that VEGF and chymase could be used as biomarkers to differentiate DF and DHF although the number of DHF samples used was low.

Data Obtained from Dengue Clinic (DC) at TTSH

Following the promising results obtained from the EDEN samples, the study was extended with the samples obtained from DC clinic. Macroglobulin was included into the list of biomarkers for analysis. The results were presented in the following three formats:
i. Using DHF as an outcome
ii. Using Severe Dengue as an outcome
iii. Using Warning Signs (WS) as an outcome

### (i) Using DHF as an Outcome

59 DF sera samples and 41 DHF sera samples were obtained from DC clinic. There was no significant differences (P>0.05) in the levels of VEGF between DF and DHF serum samples. On the other hand, significantly higher levels of macroglobulin (P<0.05) were observed in DHF samples compared to that of DF samples at both early and late time points. Chymase levels were significantly increased (P<0.05) in DHF sera samples only at the early time point. The results are shown in FIGS. 2A-2C and the summary of median, range and standard deviation obtained for various biomarkers are shown in Table 6.

### Conclusion

The data obtained from DC samples highlighted that chymase and macroglobulin can be used to differentiate DF and DHF.

### (ii) Using Warning Signs (WS) as an Outcome

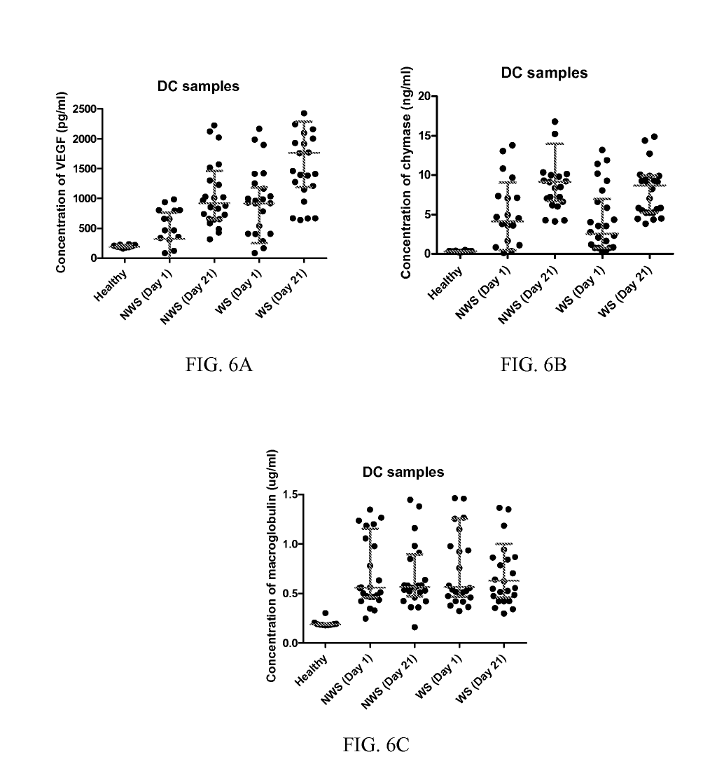

There were no significant differences (P>0.05) in the levels of chymase and macroglobulin between DF with and without warning signs (FIGS. 6A-6C & Table 7). On the other hand, comparatively higher levels of VEGF (P<0.053) was observed in DF samples with warning signs at Day 1 compared to that of DF without warning signs. Significantly higher levels of VEGF (P<0.01) were observed in DF samples with warning signs at Day 21 compared to that with without warning signs. Unfortunately, the other biomarkers such as chymase and macroglobulin could not differentiate DF with and without warning signs.

### Conclusion

The data obtained from DC samples indicated that VEGF can be used to differentiate DF with warning signs (WS) and no warning signs (NWS).

(iii) Using Severe Dengue as an Outcome

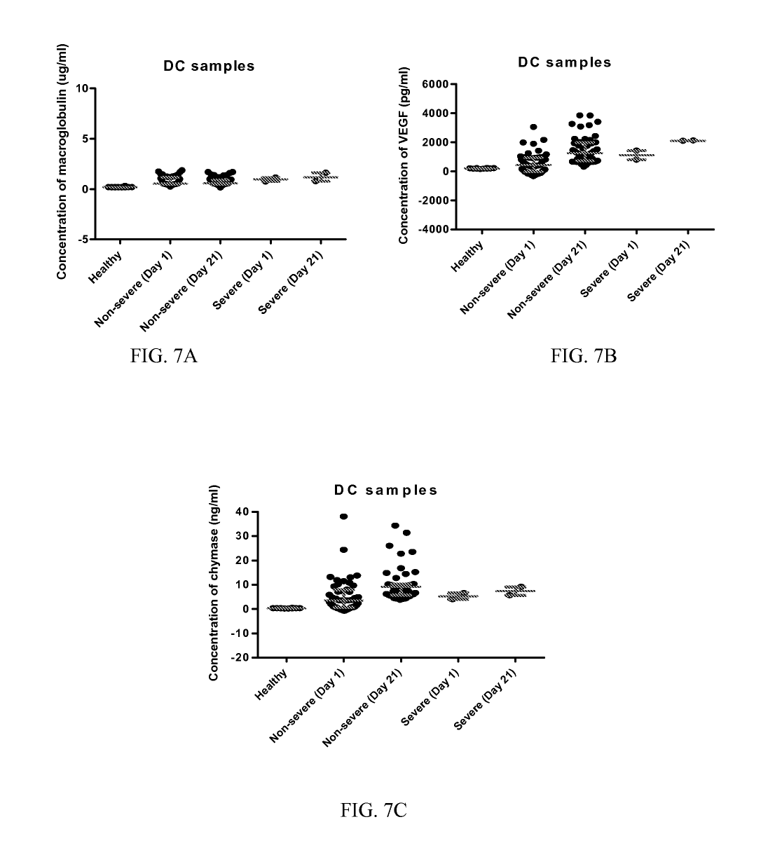

There was no significant differences (P>0.05) in the levels of chymase and macroglobulin between severe and non-severe Dengue (FIGS. 7A-7C & Table 8). On the other hand, significantly higher levels of VEGF (P<0.05) were observed in severe Dengue at day 21.

### Conclusion

The data obtained from DC samples showed that VEGF can be used to differentiate severe and non-severe Dengue at late timings.

### Analysis by TTSH Bio-Statistician

The data obtained for day 1 samples (DC samples) were re-analysed by an experienced bio-statistician (TTSH). Re-analysis was performed for three biomarkers namely VEGF, chymase and macroglobulin as they provided the promising outcome.

### Conclusion

These analyses confirmed that macroglobulin alone or in combination with chymase can be used as a biomarker to differentiate DF and DHF. Macroglobulin alone or in combination with chymase showed a sensitivity and specificity of more than 0.9. This high sensitivity and specificity of the test ensures that these biomarkers can be used. These analyses confirmed that VEGF can be used as a biomarker to differentiate between severe and non-severe dengue or DF with and without warning signs.

### Discussion and Significance

Currently, there are no reliable prognostic biomarkers to differentiate DHF/DSS from DF fever. As a result, physicians indiscriminately hospitalize all Dengue patients based on platelet count in case potentially lethal DHF/DSS later develops. This leads to an unnecessary shortage of hospital beds and significantly burdens hospital resources, especially during major epidemics. Identification of biomarkers related to disease outcome can assist the clinicians to recommend appropriate supportive care/treatment at the early stages. Moreover, the existence of credible predictive biomarkers to differentiate between DF and DHF would lead to saving of hospitalization cost.

Interestingly, the current data demonstrated that macroglobulin alone or in combination with chymase can be used as a biomarker to differentiate DF and DHF patients (specificity and sensitivity in the range of 0.9). This gives the assurance that these biomarkers are reliable potential candidates to identify DHF/DSS patients.

### REFERENCES

1. Gubler, D. J. (1998) Dengue and dengue hemorrhagic fever. Clin Microbiol Rev 11, 480-496.
2. Halstead, S. B. (2007) Dengue. Lancet 370, 1644-1652.
3. Leong, A. S., Wong, K. T., Leong, T. Y., Tan, P. H., and Wannakrairot, P. (2007) The pathology of dengue hemorrhagic fever. Semin Diagn Pathol 24, 227-236.
4. Gubler, D. J. (2011) Dengue, Urbanization and Globalization: The Unholy Trinity of the 21(st) Century. Tropical medicine and health 39, 3-11.
5. Lin, C. F., Wan, S. W., Cheng, H. J., Lei, H. Y., and Lin, Y. S. (2006) Autoimmune pathogenesis in dengue virus infection. Viral immunology 19, 127-132.
6. Clyde, K., Kyle, J. L., and Harris, E. (2006) Recent advances in deciphering viral and host determinants of dengue virus replication and pathogenesis. Journal of virology 80, 11418-11431.
7. Green, S., and Rothman, A. (2006) Immunopathological mechanisms in dengue and dengue hemorrhagic fever. Current opinion in infectious diseases 19, 429-436.
8. Halstead, S. B. (2003) Neutralization and antibody-dependent enhancement of dengue viruses. Advances in virus research 60, 421-467.
9. Pang, T., Cardosa, M. J., and Guzman, M. G. (2007) Of cascades and perfect storms: the immunopathogenesis of dengue haemorrhagic fever-dengue shock syndrome (DHF/DSS). Immunology and cell biology 85, 43-45.
10. Lin, Y. S., Yeh, T. M., Lin, C. F., Wan, S. W., Chuang, Y. C., Hsu, T. K., Liu, H. S., Liu, C. C., Anderson, R., and Lei, H. Y. (2011) Molecular mimicry between virus and host and its implications for dengue disease pathogenesis. Exp Biol Med (Maywood) 236, 515-523.
11. Liu, I. J., Chiu, C. Y., Chen, Y. C., and Wu, H. C. (2011) Molecular mimicry of human endothelial cell antigen by autoantibodies to nonstructural protein 1 of dengue virus. The Journal of Biological Chemistry 286, 9726-9736.

TABLE 4
Patient characteristics (Retrospective EDEN study)

| Characteristic | DF (n = 99) | | DHF (n = 16) | |
|---|---|---|---|---|
| Age | 38.9 | (18-66) | 45.75 | (24-77) |
| Gender | | | | |
| Male | 67 | | 9 | |
| Female | 48 | | 7 | |
| Primary/Secondary | | | | |
| infection | | | | |
| Primary | 55 | | 9 | |
| Secondary | 55 | | 7 | |
| Not classified | 5 | | — | |
| Hospitalization | | | | |
| Yes | 61 | | 16 | |
| No | 54 | | — | |
| Dengue RT-PCR | | | | |
| Positive | 110 | | 16 | |
| Negative | 5 | | — | |
| Body temperature | >38° C. | | >38° C. | |
| Complete blood count | | | | |
| Hemoglobin (g/dL) | 14.61 | (5-20.7) | 14.18 | (11-16.6) |
| Hematocrit (%) | 43.34 | (15.1-60.1) | 41.92 | (33.5-49.7) |
| White blood cell | 4.04 | (1-14.6) | 3.84 | (1.8-8.7) |
| (10³/uL) | | | | |
| Red blood cell (10⁶/uL) | 5.11 | (1.71-7.26) | 4.86 | (3.74-5.56) |
| Mean Corpuscular | 85.22 | (58.4-95.6) | 86.53 | (67.5-98.7) |
| Volume (fL) | | | | |
| Mean Corpuscular | 28.75 | (17.6-33.3) | 29.27 | (22.2-33.4) |
| Hemoglobin (pg) | | | | |
| Mean Corpuscular | 33.68 | (30.2-37) | 33.81 | (32.8-35.5) |
| Hemoglobin | | | | |
| Concentration (g/dL) | | | | |
| Platelets (10³/uL) | 165.24 | (17-400) | 142.75 | (53-276) |
| Differential WBC count | | | | |
| Neutrophils (%) | 67.04 | (−1-96.8) | 72.02 | (−1-96.9) |
| Lymphocyte (%) | 16.93 | (2-40.8) | 12.42 | (−1-32.1 |
| Mixed (%) | 5.72 | (−1-17.6) | 4.65 | (−1-14.6) |
| Miscellaneous | | | | |
| Coefficient Variation of | 25.65 | (12.2-46.5) | 18.91 | (12.4-42) |
| Red Cell Distribution | | | | |
| Width (%) | | | | |
| Standard Deviation in | 29.60 | (12-49.2) | 37.15 | (13.2-46.5) |
| Red Cell Distribution | | | | |
| Width (fL) | | | | |
| Platelet Distribution | 11.82 | (−1-18.8) | 12.16 | (−1-20.1) |
| Width (fL) | | | | |
| Mean Platelet Volume | 10.52 | (−1-14) | 10.51 | (−1-13.9) |
| (fL) | | | | |
| Platelet Larger Cell | 32.38 | (−1-60.8) | 32.31 | (−1-57.7) |
| Ratio (%) | | | | |

TABLE 5
Patient characteristics [Prospective
Adult Dengue Study (Dengue Clinic)]

| Characteristic | DF (n = 59) | | DHF (n = 41) | |
|---|---|---|---|---|
| Age | 33.84 | (20-63) | 41.95 | (21-74) |
| Dengue RT-PCR | | | | |
| Positive | 28 | | 18 | |
| Negative | 3 | | 1 | |
| Dengue NS1 antigen | | | | |
| Positive | 29 | | 17 | |
| Negative | 2 | | 2 | |
| Dengue IgM | | | | |
| Positive | 18 | | 9 | |
| Negative | 13 | | 10 | |
| Dengue IgG Indirect | | | | |
| Positive | 19 | | 14 | |
| Negative | 12 | | 5 | |
| Dengue IgG Capture | | | | |
| Positive | 5 | | 5 | |
| Negative | 26 | | 14 | |
| Body temperature | 37.50 | (36-39.3) | 37.61 | (36.5-39) |
| Duration of fever until | 4.32 | (2-5) | 4.53 | (3-5) |
| admission (Days) | | | | |
| Pulse rate (rate/min) | 76.68 | (45-100) | 75.63 | (55-90) |
| Systolic blood pressure - | 114.55 | (96-139) | 114.63 | (93-140) |
| Supine (mmHg) | | | | |
| Diastolic blood pressure - | 70.13 | (56-90) | 69.21 | (55-85) |
| Supine (mmHg) | | | | |
| Systolic blood | 109.26 | (95-137) | 110.74 | (89-130) |
| pressure - - (mmHg) | | | | |
| Diastolic blood pressure - | 69.65 | (52-86) | 70.32 | (50-89) |
| Erect (mmHg) | | | | |
| Bleeding | | | | |
| Yes | 9 | | 7 | |
| No | 22 | | 12 | |
| Fluid accumulation | No | | No | |
| Complete blood count | | | | |
| Hemoglobin (g/dL) | 15.26 | (12.6-17.2) | 14.70 | (11.6-16.4) |
| Hematocrit (%) | 44.44 | (36.5-50.1) | 42.88 | (34.7-47.4) |
| White blood cell (10³/ | 2.64 | (1.3-6.3) | 2.50 | (1.6-5.1) |
| uL) | | | | |
| Platelets (10³/uL) | 105.52 | (26-188) | 65.26 | (8-127) |
| Differential WBC count | | | | |
| Lymphocyte (%) | 26.86 | (11.7-59.9) | 23.00 | (10.9-37) |

TABLE 6
Summary of median, range and standard deviation obtained for various biomarkers (DF/DHF classification)

| | | | | | | | | | P value | |
|---|---|---|---|---|---|---|---|---|---|---|
| | | | | | | | | | (student's t test) | |
| | | | | | | | | | DF vs DHF | |
| | DF (Day 1) | | DF (Day 21) | | DHF (Day 1) | | DHF (Day 21) | | P < 0.05 | |
| | Median | | Median | | Median | | Median | | Day | Day |
| | (Range) | SD | (Range) | SD | (Range) | SD | (Range) | SD | 1 | 21 |
| VEGF | 401.44 | 705.58 | 1010.5 | 885.33 | 930.19 | 590.62 | 1339.25 | 971.10 | No | No |
| | (0-3042) | | (313-3845.5) | | (0-1979.88) | | (424.25-3836.13) | | | |
| Chymase | 0.95 | 2.70 | 9.26 | 11.22 | 8.64 | 8.70 | 7.12 | 6.71 | Yes | No |
| | (0-9.66) | | (3.8-60.15) | | (0.21-38) | | (4.25-34.32) | | | |
| Macro- | 0.49 | 0.17 | 0.52 | 0.15 | 1.24 | 0.36 | 1.06 | 0.41 | Yes | Yes |
| globulin | (0.24-1.19) | | (0.15-0.91) | | (0.36-1.86) | | (0.29-1.68) | | | |

TABLE 7
Summary of median, range and standard deviation obtained for various biomarkers (WS/NWS classification)

| | | | | | | | | | P value (stu- | |
|---|---|---|---|---|---|---|---|---|---|---|
| | | | | | | | | | dent's t test) | |
| | | | | | | | | | WS vs NWS | |
| | DF-WS (Day 1) | | DF-WS (Day 21) | | DF-NWS (Day 1) | | DF-NWS (Day 21) | | P < 0.05 | |
| | Median | | Median | | Median | | Median | | Day | Day |
| | (Range) | SD | (Range) | SD | (Range) | SD | (Range) | SD | 1 | 21 |
| VEGF | 319.25 | 652.6098 | 27569.5 | 777.2586 | 914 | 638.38 | 1761.125 | 912.152 | 0.053 | Yes |
| | (0-3042.38) | | (313-3845.5) | | (0-2161) | | (636.13-3836.13) | | | |
| Chymase | 4.15 | 8.907054 | 9.16 | 12.84293 | 2.53 | 4.20 | 8.68 | 5.134095 | No | No |
| | (0-38.05) | | (4.1-60.15) | | (0-13.175) | | (3.8-23.45) | | | |
| Macro- | 0.56 | 0.381007 | 0.57 | 0.378968 | 0.57 | 0.48 | 0.63 | 0.424248 | No | No |
| globulin | (0.24-1.52) | | (0.15-1.68) | | (0.32-1.86) | | (0.23-1.69) | | | |

TABLE 8
Summary of median, range and standard deviation obtained for various biomarkers (Severe/Non-severe) classification

| | | | | | | | | | P value | |
|---|---|---|---|---|---|---|---|---|---|---|
| | | | | | | | | | (student's t test) | |
| | Non-severe (Day 1) | | Non-severe (Day 21) | | Severe (Day 1) | | Severe (Day 21) | | P < 0.05 | |
| | Median | | Median | | Median | | Median | | Day | Day |
| | (Range) | SD | (Range) | SD | (Range) | SD | (Range) | SD | 1 | 21 |
| VEGF | 433.93 | 712.11 | 1247.37 | 921.47 | 1106.43 | 439.73 | 2104.87 | 17.67 | No | Yes |
| | (0-3042.38) | | (313-3845.5) | | (735-1417) | | (2032-2117) | | | |
| Chymase | 3.52 | 7.06 | 9.11 | 9.95 | 5.30 | 1.82 | 7.39 | 2.43 | No | No |
| | (0-38) | | (3.8-60.1) | | (4-6.6) | | (5.6-9.11) | | | |
| Macro- | 0.56 | 0.44 | 0.57 | 0.38 | 0.95 | 0.27 | 1.20 | 0.58 | No | No |
| globulin | (0.24-1.86) | | (0.15-1.69) | | (0.75-1.15) | | (0.78-1.61) | | | |

The relevant teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1. A method of determining whether a subject in need thereof has a Dengue virus infection that is likely to develop into a life-threatening Dengue disease, comprising:
(a) detecting in a sample from the subject an expression level of at least two proteins selected from the group consisting of vascular endothelial growth factor protein (SEQ ID NO:1), chymase-1 protein (SEQ ID NO:2) and alpha-2 macroglobulin protein (SEQ ID NO:3);
(b) comparing the expression level of each protein detected in (a) to a corresponding expression level for each protein in a control; and
(c) determining that the patient has a Dengue virus infection that is likely to develop into a life-threatening Dengue disease when the expression level of each protein detected in the sample in (a) is significantly greater than the corresponding expression level for each protein in the control.

2. The method of claim 1, wherein an expression level of vascular endothelial growth factor protein (SEQ ID NO:1), chymase-1 protein (SEQ ID NO:2) and alpha-2 macroglobulin protein (SEQ ID NO:3) is detected in the sample in (a).

3. The method of claim 1, wherein an expression level of vascular endothelial growth factor protein (SEQ ID NO:1) and alpha-2 macroglobulin protein (SEQ ID NO:3) is detected in the sample in (a).

4. The method of claim 1, wherein an expression level of vascular endothelial growth factor protein (SEQ ID NO:1) and chymase-1 protein (SEQ ID NO:2) is detected in the sample in (a).

5. The method of claim 1, wherein an expression level of macroglobulin protein (SEQ ID NO:) and chymase-1 protein (SEQ ID NO:2) is detected in the sample in (a).

6. The method of claim 1, wherein the expression level of each protein detected in (a) is determined using an immunoassay.

7. The method of claim 6, wherein the immunoassay is an enzyme-linked immunosorbent assay (ELISA) or lateral flow immunoassay.

8. The method of claim 1, wherein the expression level of each protein detected in (a) is determined by detecting antibodies to each protein in the sample from the subject.

9. The method of claim 1, wherein the sample is whole blood, serum, plasma, urine, saliva, tissue biopsy or a combination thereof.

10.-13. (canceled)

14. The method of claim 1, wherein the subject is known to have a Dengue virus infection.

15. The method of claim 1, wherein the subject is determined to have a Dengue virus infection that is likely to develop into a life-threatening Dengue disease before the onset of symptoms of a life-threatening Dengue disease.

16. The method of claim 1, wherein the subject is determined to have a Dengue virus infection that is likely to develop into a life-threatening Dengue disease after the onset of symptoms of a life-threatening Dengue disease.

17. A method of determining whether a subject in need thereof has a Dengue virus infection that is likely to develop into a life-threatening Dengue disease, comprising:
(a) detecting in a sample from the subject an expression level of at least two proteins selected from the group consisting of vascular endothelial growth factor protein (SEQ ID NO:1), chymase-1 protein (SEQ ID NO:2) and alpha-2 macroglobulin protein (SEQ ID NO:3);
(b) comparing the expression level of each protein detected in (a) to a corresponding expression level for each protein in a control; and
(c) determining that the patient has a Dengue virus infection that is likely to develop into a life-threatening Dengue disease when the expression level of each protein detected in the sample in (a) is significantly greater than the corresponding expression level for each protein in the control; and
(d) administering a treatment regimen for a life-threatening Dengue disease to the subject upon determining that the patient has a Dengue virus infection that is likely to develop into a life-threatening Dengue disease.

18. The method of claim 17, wherein the treatment regimen is administered to the subject before the onset of symptoms of life-threatening Dengue disease.

19. The method of claim 17, wherein the treatment regimen is administered to the subject after the onset of symptoms of life-threatening Dengue disease.

20.-34. (canceled)

35. A method of selecting a treatment regimen for a subject in need thereof who has been infected with a Dengue virus, comprising:
(a) detecting in a sample from the subject an expression level of at least two proteins selected from the group consisting of vascular endothelial growth factor protein (SEQ ID NO:1), chymase-1 protein (SEQ ID NO:2), and alpha-2 macroglobulin protein (SEQ ID NO:3);
(b) comparing the expression level of each protein detected in (a) to a corresponding expression level for each protein in a control; and
(c) selecting either:
(1) a regimen for treating a life-threatening Dengue disease when the expression level of each protein detected in the sample in (a) is significantly greater than the corresponding expression level for each protein in the control; or
(2) a regimen for treating a mild Dengue disease when the expression level of each protein detected in the sample in (a) is not significantly greater than the corresponding expression level for each protein in the control.

36. The method of claim 35, further comprising administering the regimen selected in (c) to the subject.

37.-53. (canceled)

54. A prognostic kit for distinguishing a subject who is likely to develop a mild Dengue disease from a subject who is likely to develop a life-threatening Dengue disease, comprising at least two of:
(a) one or more reagents for detecting an expression level of:
(1) a vascular endothelial growth factor protein,
(2) an antibody to a vascular endothelial growth factor protein, or
(3) a combination thereof;
(b) one or more reagents for detecting an expression level of:
(1) a chymase-1 protein,
(2) an antibody to a chymase-1 protein, or
(3) a combination thereof; or
(c) one or more reagents for detecting an expression level of:
(1) an alpha-2 macroglobulin protein,
(2) an antibody to an alpha-2 macroglobulin protein, or
(3) a combination thereof.

55. The kit of claim 54, wherein the kit comprises one or more reagents that are antibodies.

56.-60. (canceled)

61. The kit of claim 54, wherein the kit comprises one or more reagents of (a), one or more reagents of (b) and one or more reagents of (c).


SEQUENCE LISTING (3 sequences)

SEQ ID NO: 1 - protein, 302 aa, Homo sapiens
AAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKCDKPRR

SEQ ID NO: 2 - protein, 247 aa, Homo sapiens
MLLLPLPLLLFLLCSRAEAGEIIGGTECKPHSRPYMAYLEIVTSNGPSKFCGGFLIRRNFVLTAAHCAGRSITVTLGAHNITEEEDTWQKLEVIKQFRHPKYNTSTLHHDIMLLKLKEKASLTLAVGTLPFPSQFNFVPPGRMCRVAGWGRTGVLKPGSDTLQEVKLRLMDPQACSHFRDFDHNLQLCVGNPRKTKSAFKGDSGGPLLCAGVAQGIVSYGRSDAKPPAVFTRISHYRPWINQILQAN

SEQ ID NO: 3 - protein, 1474 aa, Homo sapiens
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDDEDCINRHNVYINGITYTPVSSTNEKDMYSFLEDMGLKAFTNSKIRKPKMCPQLQQYEMHGPEGLRVGFYESDVMGRGHARLVHVEEPHTETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEVKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSNHVSRTEVSSNHVLIYLDKVSNQTLSLFFTVLQDVPVRDLKPAIVKVYDYYETDEFAIAEYNAPCSKDLGNA